(19) United States
(12) Patent Application Publication
(10) Pub. No.: US 2005/0058687 A1
(43) Pub. Date: Mar. 17, 2005

(54) Covalently attached collagen VI for cell attachment and proliferation

(75) Inventors: Richard David Guarino, Holly Springs, NC (US); Jonathan A. Rowley, Chapel Hill, NC (US); Andrea Liebmann-Vinson, Willow Springs, NC (US); John Jacob Hemperly, Apex, NC (US); Mohammad A. Heidaran, Cary, NC (US); Sharon Collins Presnell, Raleigh, NC (US)

Correspondence Address: VENABLE LLP, 575 7TH STREET, N.W., WASHINGTON, DC, 20004-1601, US

(73) Assignee: Becton, Dickinson and Company, Franklin Lakes, NJ (US)

(21) Appl. No.: 10/660,781

(22) Filed: Sep. 12, 2003

(51) Int. Cl. C12N 5/02

(52) U.S. Cl. 424/426; 435/289.1

(57) ABSTRACT

Surfaces useful for cell culture comprise a support to which is bound a CAR material, and, bound to the CAR material, collagen VI or a biologically active fragment or variant thereof and, optionally, one or more other ECM proteins (or fragments or variants thereof) such as elastin, fibronectin, vitronectin, tenascin, laminin, entactin, aggrecan, decorin, collagen I, collagen III, and collagen IV. Also, optionally present on the surface is one or more polycationic polymers, such as poly-D-lysine or poly-D-ornithine. This surface is used in cell culture to promote cell attachment, survival, and/or proliferation of a number of different cell types such as (a) liver cells (e.g., HepG2 tumor cells, and a newly discovered line of rat liver epithelial stem cells) (b) osteoblasts, such as the murine cell line MC3T3 cell line and (c) primary bone marrow cells. Kits comprising the surfaces and additional reagents are also disclosed.

### BACKGROUND OF THE INVENTION

1. Field of the Invention

This invention relates generally to useful surfaces for culturing cells in vitro, and to methods for using those surfaces.

2. Description of the Background Art

Typically, for cell culture, cells are dispersed in a culture medium supplemented with serum, and the culture medium is then dispensed into a vessel that is made of a synthetic cell culture substrate such as tissue culture-grade polystyrene (PS). Under these conditions, non-specific protein adsorption to the PS surface rapidly occurs, generating a protein layer comprised of many different serum proteins in a spectrum of conformational states ranging from almost native to highly denatured. In stationary cultures, the cells subsequently settle to the surface and start to “interrogate” this poorly organized interface via cellular integrins, proteoglycans and selectins on their surface. Interactions with this randomly adsorbed protein layer lead to arbitrary biological responses that affect a variety of processes, including cell attachment (or adherence), spreading, proliferation, migration and differentiation. By contrast, in vivo, normal biological reactions occur via specific and organized ligand-receptor interactions, which in turn trigger highly organized signaling processes.

Thus, there is a need for highly defined cell culture surfaces that mimic the in vivo specificity of biological events to more effectively support desired cell biological activities during in vitro culture.

The sera conventionally used for cell culture, which includes undefined mixtures of proteins that vary from lot to lot of serum, can create further unwanted complications. For example, when cells are being prepared for in vivo uses such as cell therapy in humans, prior use of serum in culture can introduce into the cell preparation (1) biohazardous substances and (2) animal products that can induce unwanted immune responses in recipients.

Thus, there is a need for cell culture methods that employ serum free, chemically defined, culture media that provide the same benefits during culture as do sera.

The present invention is intended to meet the above needs by providing highly defined cell culture surfaces, which comprise, inter alia, the extracellular matrix (ECM) protein, collagen VI. Among the advantages of these new surfaces is that they enable the reduction of serum concentrations or the complete avoidance of serum in vitro.

### DESCRIPTION OF THE PREFERRED EMBODIMENTS

The present invention relates to a surface (such as a cell culture surface) comprising a support to which is bound a cell adhesion resistant (CAR) material and, bound to the CAR material, collagen VI or a biologically active fragment or variant thereof and, optionally, (1) one or more other ECM proteins, or biologically active fragments or variants thereof and/or (2) one or more polycationic polymers. “Biologically active” means that the fragment or variant has essentially the same activity in promoting cell attachment, survival, and/or proliferation as does the full-length, wild-type protein. “Proliferation” means that the number of cells has increased.

The present inventors found, surprisingly, that the present surfaces promote the attachment, survival and/or proliferation of a variety of cell types as well as, and often better than, standard culture surfaces using conventional conditions (e.g., incubation on conventional tissue culture PS using commercial culture media, either with or without serum). These improved effects are preferably achieved using chemically defined, serum-free media.

Also surprising is the finding that the present surfaces support such attachment, survival and/or proliferation of a wide spectrum of cell types. These include cell types found in or derived from liver, including liver tumor cells such as HepG2 (a human hepatocellular carcinoma cell line), and liver derived rat epithelial stem cells. Other cells include bone-derived cells, such as osteoblasts of the established murine cell line MC3T3 and primary rat bone marrow cells.

Advantages of this invention include:
1) The use of defined mammalian cell culture conditions, which allows the cell attachment process to be controlled by the ECM protein(s) bound to the cell culture substrate, rather than by nonspecifically (randomly and arbitrarily) adsorbed serum proteins forming a layer on the culture substrate and eliminates the need to use other uncharacterized or unpurified animal products, such as Matrigel™;
2) The ability to attribute specific cellular processes to specific ECMs, e.g., collagen VI, which eliminates the intermixed biological effects of ECM proteins with those other biological factors present in conventional serum-supplemented culture media;
3) The use of covalently bound collagen VI, either alone or with other ECM materials attached to the surface (rather than being passively adsorbed), which restricts the ECM to the substrate and prevents desorption into the liquid phase (culture medium) and also increases cell attachment by preventing solubilized ECM materials on passive coatings from blocking attachment sites on suspended cells; and
4) The ability to gain faster regulatory approval because serum is significantly reduced or eliminated, which eliminates or significantly reduces biohazardous agents, immunogenic or otherwise harmful products.

One aspect of the invention is a surface comprising (a) a support to which is bound a cell adhesion resistant (or resistive) (CAR) material, and (b) bound to the CAR material, collagen VI, or a biologically active fragment or variant thereof, and, optionally, one or more other ECM proteins, or a biologically active fragments or variants thereof. The other ECMs may be, e.g., elastin, fibronectin, vitronectin, tenascin, laminin, entactin, aggrecan, decorin, or other collagens, such as collagen I, collagen III, or collagen IV. Optionally, one or more polycationic polymer, such as polyethyleneimine (PEI), poly-D-lysine (PDL), poly-L-lysine, poly-D-ornithine (PDO) or poly-L-lysine (PLO), may also be bound to the CAR material.

Another aspect of the invention is a surface comprising (a) a support to which is bound a CAR material, and (b) bound to the CAR material, collagen VI, or a biologically active fragment or variant thereof, and one or more other ECM proteins, or a biologically active fragment or variant thereof. The other ECM proteins may be, e.g., elastin, fibronectin, vitronectin, tenascin, laminin, entactin, aggrecan, decorin, or a collagen, such as collagen I, collagen III, and/or collagen IV.

As used herein, the term “CAR material” refers to a material that, when present on a surface, prevents, inhibits, or reduces the non-specific binding (adhesion) to the support of cells, proteins or polypeptides found on cell surfaces. CAR materials are resistant to mammalian cells and preferably also to microorganisms. CAR materials are sometimes referred to as “non-fouling substrates,” “inert coatings,” “low affinity reagents,” or “non-adhesive coatings. Examples of CAR materials include hyaluronic acid (HA) or a derivative thereof, alginic acid (AA) or a derivative thereof, polyhydroxyethylmethyacrylate (poly-HEMA), polyethylene glycol (PEG), glyme or a derivative thereof, polypropylacrylamide, polyisopropylacrylamide, or a combination of these compounds. Preferably, the CAR material is HA.

In some embodiments, one or more of a proteoglycan, a biglycan, a glycosaminoglycan, or Matrigel™ may be bound to the CAR material.

A protein or other substances bound to a CAR material, for example, collagen VI, another ECM protein, or a polycationic polymer, may be bound either covalently or non-covalently, but is preferably covalently bound.

The support may be a natural or synthetic organic polymer, or an inorganic composite. Suitable supports include polystyrene (PS), polypropylene, polyethylene, polyethylene terephthalate, polytetrafluoroethylene, polylactide, cellulose, glass, or ceramic. Preferably, the support is PS.

The invention is also directed to an article of manufacture comprising a surface of the invention as described above. Examples of preferred articles are a cell culture vessel, such as a slide, a multi-well plate, a culture dish, a culture flask, a culture bottle, etc. Alternatively, the article may be part of a medical device, a scaffold or a template for generating a 3D implant, tissue and/or organ, or a foam or fiber mesh.

Another aspect of the invention is a method of making the above surface of the invention, comprising (a) attaching a CAR material to a support, and (b) attaching to the CAR material collagen VI or a biologically active fragment or variant thereof and, optionally, one or more other ECM proteins (or a biologically active fragment or variant of the ECM protein) and/or one or more polycationic polymers. Any of the ECM proteins or polycationic polymers disclosed herein, or others, may be used.

In one embodiment, the CAR material is attached to the support by treating the support with an oxidizing plasma, and binding the CAR material to the treated support. In another embodiment, the CAR material is attached to the support by treating the support with an oxidizing plasma; exposing the treated support to a polycationic polymer with amino groups to form an intermediate layer; and binding the CAR material to the intermediate layer. Preferably, the polycationic polymer is polyethylene imine (PEI) or poly-L-lysine (PLL).

Another aspect of the invention is a method for promoting the attachment, survival, and/or proliferation of a cell in culture. The method comprises, contacting the cell in a culture medium with a surface of the invention under conditions effective for the attachment, survival and/or proliferation of the cell. Examples of surfaces are those comprising (a) a support to which is bound a CAR material, and (b) bound to the CAR material, collagen VI, or a biologically active fragment or variant thereof, and, optionally, one or more other ECM proteins (or a biologically active fragment or variant thereof). Examples of preferred ECM proteins in this method include elastin, fibronectin, vitronectin, tenascin, laminin, entactin, aggrecan, decorin, and other collagens, such as collagen I, collagen III, or collagen IV. Elastin, fibronectin, vitronectin, collagen I, collagen III, and collagen IV are most preferred. Also, optionally bound to the car material is one or more polycationic polymers (e.g., PEI, PDL, PLL, PLO or PDO).

In one embodiment of the above method, the surface comprises (a) a support to which is bound a CAR material, and (b) bound to the CAR material, collagen VI, or a biologically active fragment or variant thereof, as well as one or more of the ECM proteins listed above (or a biologically active fragment or variant thereof).

Though the collagen VI and/or other ECM proteins or polycationic polymers in the above methods may be covalently or non-covalently bound to the CAR material, they are preferably covalently bound. The support material and the CAR material may be any of those noted above. A preferred support is PS and a preferred CAR material is hyaluronic acid (HA).

In preferred embodiments of this method, the cell is a mammalian cell, most preferably a human cell. Preferred cells are liver cells (including cells from a liver tumor or an established hepatocyte or liver tumor cell line such as Hep2G cells). Also included are bone cells (e.g., osteoblasts such as the MC3T3 cell line) and bone marrow cells. The cell may be an epithelial stem cell, such as a liver epithelial stem cell. Rat liver epithelial cells are described herein.

In embodiments of this method, the culture medium may be supplemented with serum, but is preferably serum-free. A suitable chemically defined serum free media—BD Medium #1—is described herein.

This method may also be used in drug discovery, for example, to identify a potential drug target, to determine the effect of an agent on a property of the cell, or to determine if a potential agent is toxic to the cell, etc. Another aspect of the invention is a method for identifying a factor in a test sample that stimulates or inhibits proliferation of cells in culture, comprising (a) contacting the cells in a serum-free culture medium with a surface of the invention and with the test sample suspected of including the factor, and (b) measuring the proliferation of these cells compared to proliferation of similar control cells without the test sample. Increased proliferation in the presence of the test sample indicates the presence of a factor that stimulates cell proliferation of the cell, and decreased proliferation in the presence of the test sample indicates the presence of a factor that inhibits cell proliferation of the cell. A similar method may be used wherein the outcome measure is cell attachment, or cell survival using appropriate and known methods to measure each of these classes of responses.

Also provided is a kit useful for promoting the attachment, survival, and/or proliferation of a cell, comprising a surface of the invention and one or more components or reagents suitable for culturing the cells and enabling cell attachment, survival, and/or proliferation. Another kit embodiment, useful for identifying a factor that modulates cell attachment, survival and/or proliferation (or any of the other cell behaviors) in culture, comprising a surface of the invention and one or more components or reagents suitable for (a) attaching, growing or promoting survival of the cells and (b) measuring the cell's attachment, survival and/or proliferation is also provided for herein.

### BRIEF DESCRIPTION OF THE DRAWINGS

FIG. 1 shows proliferation and attachment studies with Hep G2 cells.

FIG. 2 shows studies of the proliferation of Hep G2 cells.

FIG. 3 shows proliferation and attachment studies with rat epithelial stem cells.

FIG. 4 shows studies of the proliferation of rat epithelial stem cells.

FIG. 5 shows proliferation and attachment studies with MC3T3 osteoblast cells.

FIG. 6 shows proliferation and attachment studies with rat bone marrow cells.

### DETAILED DESCRIPTION OF THE INVENTION

Surfaces of the invention comprise a solid, preferably polymeric, support, to which is bound a CAR material. The support may take any of a variety of forms. It may be of any suitable shape, such as those used for cell culture vessels (a slide, multi-well plate, culture dish, etc.) and may be two- or three-dimensional. It may be any of a variety materials, including natural polymers, synthetic polymers and inorganic composites. Natural polymers include, e.g., collagen-and glycosaminoglycan (GAG)-based materials. Synthetic polymers include, e.g., poly(a-hydroxy acids) such as polylactic acid (PLA), polyglycolic acid (PGA) and copolymers thereof (PLGA), poly(ortho ester), polyurethanes, and hydrogels, such as polyhydroxyethylmethacrylate (poly-HEMA) or polyethylene oxide-polypropylene oxide copolymer. Hybrid materials, containing naturally derived and synthetic polymer materials, may also be used. Non-limiting examples of such materials are disclosed in Chen et al. (2000), Advanced Materials 12:455-457. Inorganic composites include, e.g., calcium phosphate ceramics, bioglasses and bioactive glass-ceramics, in particular composites combining calcium hydroxyapatite and silicon stabilized tricalcium phosphate. Among preferred supports are PS, polypropylene, polyethylene, polyethylene terephthalate, polytri- or tetra-fluoroethylene, polyhexafluoropropylene, polyvinyl chloride, polyvinylidine fluoride, polylactide, cellulose, glass, or a ceramic. In a preferred embodiment, the support is part of a tissue culture vessel, such as a PS tissue culture dish or multi-well plate.

Any suitable CAR material, many of which are known to those skilled in the art, may be bound to the support. Typical CAR materials include hyaluronic acid (HA) or a derivative thereof, alginic acid (AA) or a derivative thereof, poly-HEMA, polyethylene glycol (PEG), glyme or a derivative thereof, polypropylacrylamide, and polyisopropylacrylamide, or a combination of these materials. In a preferred embodiment, the CAR material is HA.

The CAR material is preferably bound to the support by covalent bonds. Various types of covalent bonds can form, some of which are discussed in more detail in co-pending, commonly assigned U.S. patent application Ser. No. 10/259,797 by Andrea Liebmann-Vinson and R. Clark, filed Sep. 30, 2002; U.S. patent application Ser. No. 10/260,737 by Mohammad A. Heidaran and Mary K. Meyer entitled Method and Apparatuses for the Integrated Discovery of Cell Culture Environments, filed Sep. 30, 2003; U.S. patent application Ser. No. 10/259,815 by John J. Hemperly, entitled Proliferation and Differentiation of Stem Cell from Bone Marrow and Other Cells Using Extracellular Matrix and other Molecules, filed Sep. 30, 2002; and attorney-docket number 7767-184045, filed Aug. 15, 2003 which are incorporated herein by reference. These applications also disclose other aspects of making and using surfaces that include supports with bound CAR materials and ECM proteins.

In one embodiment, collagen VI (or a biologically active fragment or variant thereof) and, optionally, one or more additional ECM proteins (or a biologically active fragment or variant thereof) and/or one or more polycationic polymer are bound to the CAR material. In a preferred embodiment, collagen VI (or a biologically active fragment or variant thereof) and one or more other ECM proteins (or a biologically active fragment or variant thereof) are bound to the CAR material.

The collagen VI or, optionally, additional ECM protein(s) can be in the form of a naturally occurring polypeptide (protein), a recombinant polypeptide, or a synthetic or semi-synthetic polypeptide, or any combination thereof. The terms “polypeptide” and “protein” are used interchangeably herein.

Methods of cloning, expressing and purifying polypeptides, such as ECM proteins, are conventional, as are methods of generating synthetic or semi-synthetic polypeptides. ECM proteins can also be obtained from commercial sources.

Biologically active fragments or variants of collagen VI or, optionally, one or more other ECM proteins may be bound to the CAR surface along with the collagen VI. As used herein, the term “a biologically active fragment or variant thereof” includes a polypeptide that retains substantially at least one of the biological functions or activities of the wild type polypeptide. For example, a biologically active fragment or variant of collagen VI (or other ECM protein or polycationic polymer) is one that can bind to a CAR material, while retaining the ability to promote the attachment, survival, and/or proliferation of a cell when used in a method of the invention.

Biologically active fragments can be of any size that is compatible with their requisite activity ranging from a polypeptide that is shortened at the N-terminus or C-terminus by only 1 or 2 amino acids to a peptide having between about 3-20 amino acids. Those skilled in the art can readily determine if a given fragment retains a desired biological activity using methods described herein or methods well known in the art. An example of a biologically active fragment is the extracellular domain of an ECM protein, which retains its ability to bind cells.

Biologically active variants can take a variety of forms. For example, one or more of the amino acid residues may be substituted with a conserved or non-conserved amino acid residue (preferably a conserved amino acid residue). A variant can differ in amino acid sequence from the wild type polypeptide by, e.g., one or more additions, substitutions, deletions, insertions, inversions, fusions, and truncations, or a combination of any of these. Other active variants, many of which will be evident to the skilled worker, include polypeptides that are conjugated to another compound or fused to another, possibly heterologous, peptide sequence.

Preferred ECM proteins for binding to a CAR surface and used herein include elastin, fibronectin, vitronectin, tenascin, laminin, entactin, aggrecan, decorin, and collagens, such as collagen I, collagen III, or collagen IV. The Examples herein illustrate the use of a variety of combinations of collagen VI and, optionally, other ECM proteins or polycationic polymers in methods of the invention. Other compounds that can be bound to CAR materials include proteoglycans, biglycans, glycosaminoglycans, and/or Matrigel™.

Collagen VI and/or other ECM proteins or polycationic polymer can be bound to the CAR material either covalently or non-covalently (e.g., passively adsorbed, such as by electrostatic forces, ionic or hydrogen bonds, hydrophilic or hydrophobic interactions, Van der Waals forces, etc.). In a preferred embodiment, the binding is covalent. Co-pending U.S. patent applications Ser. No. 10/259,797, 10/260,737 and 10/259,815 describe such covalent binding of molecules to CAR surfaces.

Methods of making surfaces in which a CAR material is bound to a support, and in which ECM proteins, polycationic polymers, or the like, are bound to the CAR material, are described in detail in co-pending U.S. patent applications Ser. No. 10/259,797, 10/260,737 and 10/259,815. In brief, one method of attaching a CAR material to a support comprises treating the support with an oxidizing plasma, and binding the CAR material to the treated support. Another method of attaching a CAR material to a support comprises treating the support with an oxidizing plasma; exposing the treated support to a polycationic polymer with amino groups (such as PEI, PLL, poly-D-lysine (PDL), poly-L-ornithine (PLO), poly-D-ornithine (PDO), poly(vinylamine) (PVA) or poly(allylamine) (PAA), preferably, PEI or PLL) to form an intermediate layer, and binding the CAR material to the intermediate layer. Methods of binding an ECM or a polycationic polyaminoacid to a CAR material are conventional. These include, e.g., sodium periodate oxidation and reductive amination, etc.

A variety of articles may comprise a surface of the invention. Suitable articles will be evident to those of skill in the art. Such articles include cell culture vessels, such as slides (e.g., tissue slides, microscope slides, etc.), plates (e.g., culture plates or multi-well plates, including microplates), flasks (e.g., stationary or spinner flasks), bottles (e.g., roller bottles), bioreactors, or the like. Other suitable articles are medical devices, such as extracorporeal devices, artificial joints, and liver assist devices. Others are tubes, sutures, membranes, films, microparticles (preferably made of plastic) and scaffolds or other templates for generating two- or three-dimensional implants, tissues and/or organs. In one embodiment, such a scaffold or template is seeded with cells and then implanted into a suitable location in the body of a mammal. In another embodiment, the scaffold is implanted into a subject, and cells are allowed to attach to it at the site of implantation. Articles such as scaffolds or templates may be any suitable material, e.g., glass, plastic, foam or fiber mesh.

The invention relates to a method of promoting the attachment, survival, and/or proliferation of a cell in culture, comprising contacting the cell in a culture medium with a surface of the present invention. Cell “attachment” means binding of the cell to the surface such that the cell is not eluted by conventional washing or handling procedures. By cell “survival,” particularly a primary cell, is meant sustained viability. “Proliferation” means an increase in the cell number.

The cell may be “contacted” or brought into contact with the surface by any suitable means. For example, cells in a culture medium may be poured, pipetted, dispensed, etc., into a culture vessel comprising the surface, or a medical device or scaffold comprising the surface may be submerged in culture medium in which the cells are suspended.

Any of the inventive surfaces described herein is suitable for this method. In one embodiment, the surface comprises collagen VI bound to HA and, optionally, one or more further ECM proteins and/or a polycationic polymer. In another embodiment, at least one additional ECM protein is included. In a preferred embodiment, the support is PS, the CAR material is HA, to which may be bound one or more of the other ECM protein(s), such as elastin, fibronectin, vitronectin, collagen I, collagen III and collagen IV. The Examples below describe the use of some typical combinations of collagen VI and other ECM proteins or polycationic polymers in the present methods. Of course, other combinations can also be used.

A variety of cell types may be cultured by methods of the invention. Any cell, including plant, yeast or mammalian cells, that can be cultured in vitro may be used. Particularly well-suited to the methods of the invention are mammalian cells. Human cells are most preferred. For example, the Examples herein illustrate the culture of: liver-derived cells (HepG2 cells), a human hepatoma carcinoma cell line (ATCC HB-8065) and bone-derived MC3T3 osteoblasts. Primary rat bone marrow cells are also illustrated. Other cell types, such as epithelial stem cells derived from liver or other tissues, and other primary human cells (e.g., autologous cells or cells from a donor that are intended for transplantation into a subject, preferably liver cells), can also be cultured by methods of the invention. Table 1 illustrates the ability of surfaces of the invention to support attachment, survival, and proliferation of various cell types.

TABLE 1

| | Attachment | Survival | Proliferation |
|---|---|---|---|
| Collagen VI | HepG2, RESC, RBMC, | HepG2, RESC, | HepG2, RESC, |
| alone | MC3T3 | RBMC, MC3T3 | RBMC, MC3T3 |
| Col. VI + collagen I | HepG2, RESC, RBMC, | HepG2, RESC, | HepG2, RESC, |
| | MC3T3 | MC3T3 | MC3T3 |
| Col. VI + collagen | HepG2, RESC, RBMC, | HepG2, RESC, | MC3T3 |
| III | MC3T3 | RBMC, MC3T3 | |
| Col. VI + collagen | HepG2, RESC, RBMC, | HepG2, RESC, | HepG2, RESC, |
| IV | MC3T3 | RBMC, MC3T3 | MC3T3 |
| Col. VI + elastin | HepG2, RESC, RBMC, | HepG2, RESC, | HepG2, RESC, |
| | MC3T3 | RBMC, MC3T3 | MC3T3 |
| Col. VI + fibronectin | HepG2, RESC, RBMC, | HepG2, RESC, | HepG2, RESC, |
| | MC3T3 | RBMC, MC3T3 | MC3T3 |
| Col. VI + laminin | HepG2, RESC, RBMC, | HepG2, RESC, | HepG2, RESC, MC3T3 |
| | MC3T3 | MC3T3 | |
| Col. VI + vitronectin | HepG2, RESC, RBMC, | HepG2, RESC, | HepG2, RESC, |
| | MC3T3 | RBMC, MC3T3 | RBMC, MC3T3 |
| Col. VI + poly- | HepG2, MC3T3 | HepG2, RESC, | MC3T3 |
| L- | | RBMC, MC3T3 | |
| ornithine | | | |
| Col. VI + Poly- | HepG2, MC3T3 | HepG2, RESC, MC3T3 | MC3T3 |
| D-lysine | | | |

Examples III and IV include studies with a rat liver epithelial stem cell line that was derived and characterized by the present inventors. This cell line is similar to those described in “Liver Growth and Repair” edited by A. J. Strain and A. M. Diehl, pp 68-71, Chapman and Hall, 1998. See also Grisham, J. W., Thal, S. B. And Nagel, A. (1975). Cellular derivation of continuously cultured epithelial cells from normal rat liver, in Gene Expression and Carcinogeneisis in Cultured Liver (Eds. L. E. Gerschenson and B. B. Thompson, Academic Press, New York, pp. 1-23.)

Any of a variety of culture media may be used in conjunction with the inventive surfaces in the present methods. Commercially available media, such as DMEM, F12, αMEM, Hepatostim™, RPMI, or combinations thereof, may be used, either in the presence or absence or serum. Suitable sera include calf serum, fetal calf serum, horse serum, or the like. Preferably, a synthetic, chemically-defined, serum-free medium is used. A variety of suitable chemically defined media will be evident to the skilled worker. One such media, BD Medium 1, is employed in the Examples. The composition of BD Medium 1 is summarized in Table 2.

TABLE 2
BD Medium #1

| | Concentration | Concentration |
|---|---|---|
| | (% or molarity) | (Mg/L) |
| Media components: | | |
| Albumin, bovine serum | 0.1% | 0.2 |
| Calcium Chloride (CaCl₂.2H₂O) | 1.636 mM (as | 220.5 |
| | are others) | |
| Cupric Sulfate (CuS0₄.5H₂O) | 10E−06 | 0.00025 |
| Ferric Nitrate (Fe(N0₃)3.9H₂O) | 0.001 | 0.404 |
| D-Glucose: same as base media | 11.8964 | 3603 |
| Glutathione Reduced | 2 | 614.6 |
| Magnesium chloride (MgCl₂.6H₂O) | 0.5 | 101.65 |
| L-Asparagine (anhydrous) | 0.2 | 26.42 |
| L-Cysteine-(free base) | 0.5 | 60.6 |
| L-Glutamine | 1 | 146.1 |
| 4-hydroxy-L-proline | 0.2 | 26.22 |
| L-Leucine | 0.5 | 65.6 |
| L-Proline | 0.2 | 23.02 |
| L-Serine | 0.5 | 52.55 |
| Putrescine9HCl | 0.1 | 16.11 |
| Retinol acetate (Vitamin A) | 0.00031 | 0.10004 |
| Sodium selenite | 2.9E−05 | 0.00501 |
| Thymidine | 0.0025 | 0.6055 |
| Zinc sulfate (ZnS0₄.7H₂O) | 0.003 | 0.8625 |
| Vitamins: 10 items | | |
| d-Biotin (Vit H) | 0.0017 | 0.41531 |
| Choline Chloride | 0.0369 | 5.15124 |
| Folk Acid (pteroylglutamic acid) | 0.0056 | 2.56012 |
| myo-inositol | 0.0717 | 12.9203 |
| Nicotinamide | 0.0119 | 1.45299 |
| PABA (p-Aminobenzoic Acid) | 0.0072 | 0.96764 |
| DL-Pantothenic Acid Hemicalcium Salt | 0.004 | 1 |
| Pyridoxal | 0.0055 | 1.234 |
| Riboflavin (Vit B2) | 0.0004 | 0.15056 |
| Thiamine (Vit B1) (aneurine HCl) | 0.0007 | 0.23611 |
| Essential Amino Acids: 11 items | | |
| L-Arginine HCl | 0.906 | 190.9 |
| L-Cystine | 0.42 | 62.64 |
| L-Histidine | 0.2643 | 59.53 |
| L-Isoleucine | 0.5673 | 74.39 |
| L-Lysine HCl | 0.419 | 76.51 |
| L-Methionine | 0.2374 | 35.36 |
| L-Phenylalanine | 0.2604 | 46.26 |
| L-Threonine | 0.431 | 51.36 |
| Tryptophan | 0.0605 | 12.46 |
| L-Tyrosine 2Na*2H₂O | 0.3249 | 56.69 |
| L-Valine | 0.4105 | 46.13 |
| Magnesium Sulfate (H₂SO₄) | 0.6102 | 10 |
| Potassium Chloride | 4.7709 | 400 |
| Sodium Bicarbonate (NaHC0₃) | 26.146 | 2200 |
| Sodium Chloride | 113.599 | 12676 |
| Sodium Phosphate Monobasic | 1.7706 | 125 |
| NaH₂PO₄ (anhydrous) | | |

In the above methods, a cell is contacted with a surface of the invention under conditions effective for the attachment, survival and/or proliferation of the cell. By “effective” conditions is meant conditions that result in a measurable amount of cell attachment, survival and/or proliferation. Effective conditions can be readily determined and/or optimized by a skilled worker, using conventional methods. Among the factors to be varied include, e.g., the seeding density, the vessel, culture medium, temperature, O₂/CO₂ concentrations, and the like. Some typical effective conditions are described in the Examples.

Another aspect of the invention is a method for identifying a test sample containing an agent (factor) that modulates (e.g., stimulates, inhibits, potentiates, etc.) proliferation of a cell in culture, comprising (a) contacting the cell, in a culture medium lacking serum, with a surface of the invention and with the test sample suspected of including the factor, and (b) measuring the proliferation of the cell compared to proliferation of a similar cell in a culture in the absence of the test sample, wherein (i) increased proliferation in the presence of the test sample indicates the presence in the test sample of a factor that stimulates proliferation of the cell, and (ii) decreased proliferation in the presence of the test sample indicates the presence in the sample of a factor that inhibits proliferation of the cell. The comparison can be made to a cell to which the test sample has not been added, which is grown in parallel with the experimental sample; or the comparison can be made to a reference database. The test sample may be a pure compound whose effects are unknown, or a composition whose contents and effects are unknown.

One of skill in the art will recognize a variety of types of agents that can be tested in this method. For example, the method can be used to test putative drugs (e.g., proteins, peptides, small molecules, nucleic acids, such as antisense molecules, ribozymes or RNAi, or the like) that affect an activity of a cell of interest (e.g., an intercellular signaling cascade, a metabolic pathway, etc.). In addition to drug screening, drug discovery, and the identification of potential drug targets, the method can be used to determine if a potential agent is toxic to the cell and has a measurable detrimental effect, induces unregulated proliferation (oncogenic transformation), etc.

In another embodiment, the agent tested is a putative factor that can induce, enhance, or maintain a marker of interest, or that is important for the maintenance of a desirable cellular function. Typical such markers/functions that can be studied in liver cells include (1) the induction of drug/toxin metabolizing enzymes of the cytochrome P₄₅₀ family (CYP), an important hepatocyte function; or (2) the production of albumin, a function that is usually lost during primary culture of hepatocytes but which is maintained in HepG2 cells.

Among the types of agents that can be tested are proliferation factors, such as angiopoietin 2, BMP2, BMP4, erythropoietin, aFGF, bFGF, HGF, insulin, noggin, PDGF, TNF, VEGF, stem cell factors, GDF6, CSF, FH3/F2, TGFβ, or the like. Alternatively, one can test small molecules generated by conventional combinatorial chemistry, or peptide libraries. (See, for example, co pending U.S. patent applications Ser. Nos. 10/260,737 and 10/259,816.) Other types of agents will be evident to the skilled worker.

Any of the methods of the invention can be adapted to high throughput procedures. One or more of the processes may be achieved robotically.

Another aspect of the invention is a kit useful for promoting the attachment, survival, and/or proliferation of cells, comprising a surface of the invention and one or more components or reagents suitable for culturing the cell (e.g., a culture vessel, an appropriate culture medium and/or factor(s) that enhance cell proliferation, etc.).

Another kit of the invention useful for identifying a factor that modulates proliferation of a cell in culture, comprises a surface of the invention and one or more components suitable for cell culture (leading to proliferation) and for measuring cell proliferation in the culture. The components may include a culture vessel, an appropriate culture medium, factor(s) that enhance cell proliferation, and/or one or more reagents, such as those described herein, that can be used to measure cell proliferation.

Such kits have many uses, which will be evident to the skilled worker. For example, they can be used to propagate cells of interest, such as primary cells, stem cells, cells to be used in methods of cell therapy, etc., to characterize agents, such as putative therapeutic agents, to identify agents that play a role in a cell function of interest, etc. Such kits could be of commercial use, e.g., in high-throughput drug studies.

In the foregoing and in the following Examples, all temperatures are set forth in uncorrected degrees Celsius, and, unless otherwise indicated, all parts and percentages are by weight.

Having now generally described the invention, the same will be more readily understood through reference to the following examples that are provided by way of illustration, and are not intended to be limiting of the present invention, unless specified.

### EXAMPLE I

### Attachment and Proliferation of Hep G2 Human Hepatoma Cells in Serum-Free Medium

HepG2 human hepatoma cells were grown in BD Medium #1, a serum free chemically defined medium, on surfaces comprising hyaluronic acid (HA) to which was covalently attached collagen VI, either alone or in combination with other covalently attached extracellular matrix (ECM) proteins. (The components of BD Medium #1 are summarized in Table 2.) The ECM combinations tested were: collagen VI alone, or collagen VI in combination with either elastin, fibronectin, collagen I, collagen IV or vitronectin. In control samples, cells were seeded in BD Medium #1 onto standard tissue culture treated polystyrene.

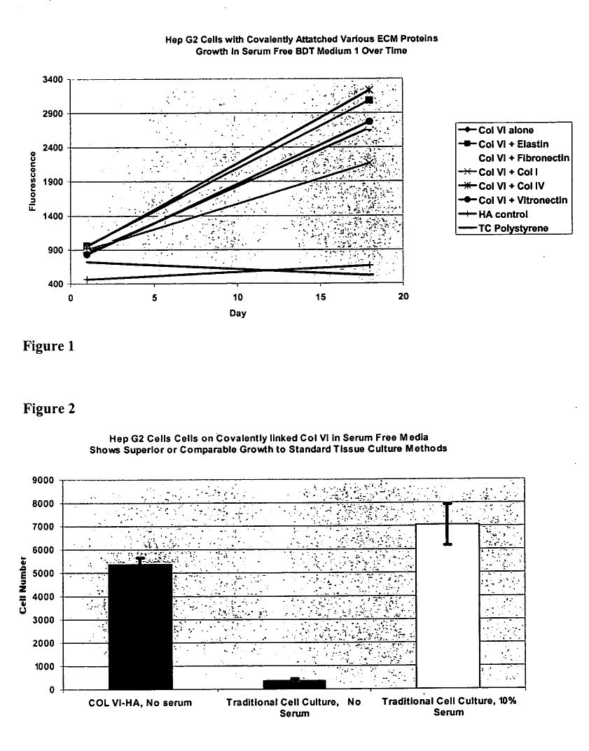

The cells were seeded in wells of 96-well microplates at an initial density of 10⁴ cells/well, incubated in a CO₂ incubator at 37° C., and stained at the time points indicated in the figure, using propidium iodide. Fluorescence was measured with a BMG Polarstar fluorometer at excitation of 544 nm and emission of 615 nm. As shown in FIG. 1, the number of cells on the surfaces coated with collagen VI increased between day 1 and day 18. Cells seeded on tissue culture polystyrene or a cell adhesion resistant surface lacking any extracellular matrix proteins did not proliferate, indicating that the presence of collagen VI alone or collagen VI combined with other extracellular matrix support proliferation in a serum-free environment.

### EXAMPLE II

### Comparison of Proliferation of Hep G2 Human Hepatoma Cells in Serum-Free Medium to Commercial Media

HepG2 human hepatoma cells were grown as described in Example I, except the only ECM covalently bound to the HA surface was collagen VI. Proliferation of the cells on this collagen VI-surface in BD Medium #1 was compared to proliferation under the standard tissue culture conditions, either with or without serum. The cell number after 5 days of culture is shown graphically in FIG. 2.

The cells were stained with propidium iodide. Fluorescence microscopy images were obtained on an HT Imager (Discovery-1, Universal Imaging Corporation, a subsidiary of Molecular Devices, Downington, Pa.) at excitation of 535 nm and emission of 700 nm. Cell numbers were determined from these fluorescence microscopy images using UIC Metamorph™ analysis software. FIG. 2 shows that collagen VI combined with serum free BD Medium #1 promoted the proliferation of Hep G2 cells to the same extent as the standard tissue culture conditions (tissue culture PS surfaces, DMEM) with serum, and was vastly superior to the standard culture conditions with no serum.

### EXAMPLE III

### Attachment and Proliferation of Rat Epithelial Stem Cells in Serum-Free Medium

Rat epithelial stem cells (passage 6) were grown in BD Medium #1 on surfaces comprising hyaluronic acid (HA) to which was covalently attached collagen VI alone, or in combination with either elastin, fibronectin, collagen I, collagen IV vitronectin, or collagen III. Control samples were (1) cultured under “standard tissue culture conditions,” which comprised seeding cells onto tissue culture PS plates using commercial medium (DMEM/F12 mixed 1:1), or (2) cultured on a hyaluronic acid (HA) surface with no extracellular matrix protein present in BD Medium #1. The cells were stained with propidium iodide and analyzed as described in Example II. The proliferation over time was assayed.

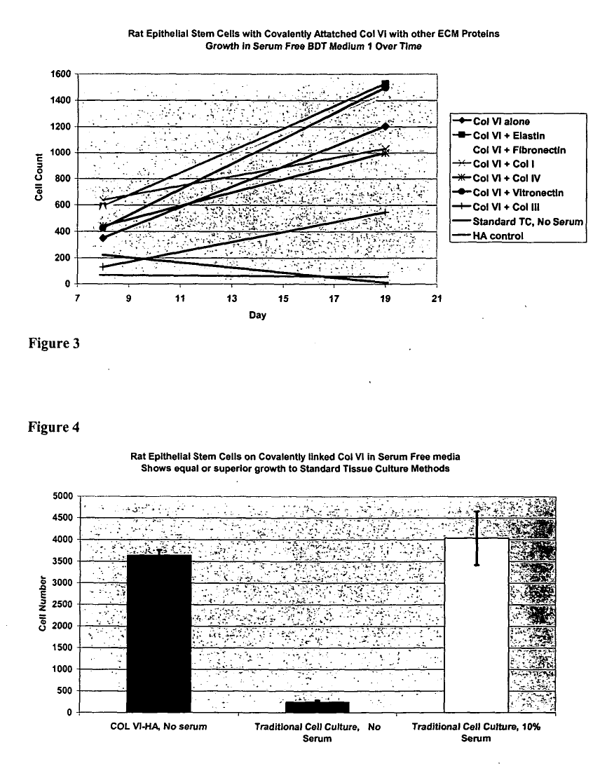

As shown in FIG. 3, the number of cells on the collagen VI surfaces increased between day 8 and day 19. The proliferation in BD Medium #1 on the surfaces comprising collagen VI was superior to proliferation in commercial media with no serum. The absence of proliferation on the HA control demonstrates that the collagen VI alone or in combination with other extracellular matrix proteins allowed for efficient attachment and proliferation of the rat epithelial stem cells.

### EXAMPLE IV

### Comparison of Proliferation of Rat Epithelial Stem Cells in Serum-Free Medium to Commercial Media

Rat epithelial stem cells (passage 9) were grown as described in Example III, except the only ECM covalently bound to the HA surface was collagen VI. Proliferation of the cells on the collagen VI surfaces in BD Medium #1 was compared to proliferation in the standard tissue culture conditions, either with or without serum. The cells were stained with propidium iodide and analyzed as described in Example II. As shown in FIG. 4, collagen VI combined BD Medium #1 promoted the proliferation of the rat epithelial stem cells to the same extent as did standard tissue culture conditions (tissue culture PS surfaces, DMEM) with serum, and was superior to proliferation using the standard conditions with no serum.

### EXAMPLE V

### Proliferation and Attachment of MC3T3 Osteoblasts

MC3T3 osteoblast cells were grown in commercial αMEM (Gibco/Invitrogen) with 10% serum, on surfaces comprising hyaluronic acid (HA) to which was covalently attached Collagen VI, either alone or in combination with either elastin, fibronectin, collagen III, vitronectin, poly-D-lysine (PDL), poly-D-ornithine (PDO), collagen IV, collagen I, or laminin. The proliferation on covalently linked extracellular matrix protein was compared to the proliferation under standard tissue culture conditions at 12, 72 and 120 hours, and is shown in FIG. 5. Cells were stained with propidium iodide and well fluorescence was measured with a BMG Polarstar fluorometer at excitation of 544 and emission of 614 (gain=40). The figure shows that the covalently linked collagen VI, either alone or in combination with other covalently attached ECM proteins, is important for efficient cell adhesion and proliferation of the bone cell-derived MC3T3 cells.

### EXAMPLE VI

### Proliferation and Attachment of Primary Rat Bone Marrow Cells in Serum-Free Medium

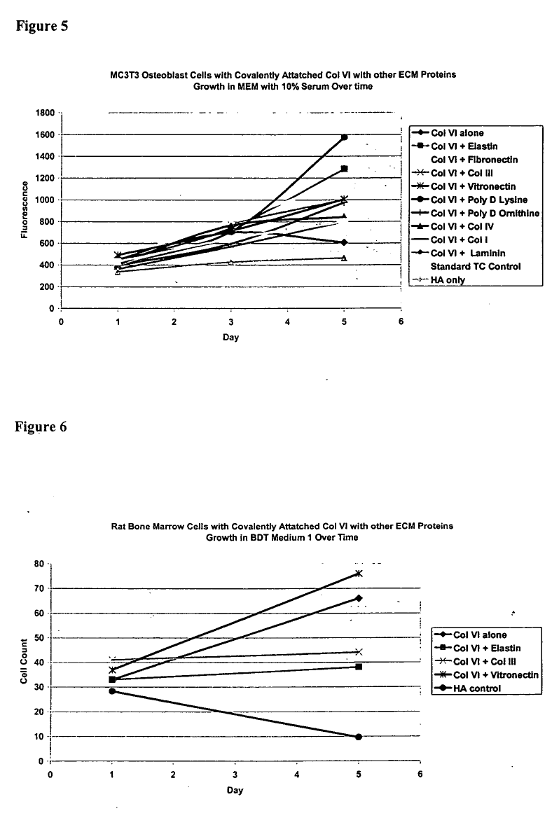

Rat bone marrow cells were isolated and plated in tissue culture flasks, and fed twice with DMEM supplemented with 10% fetal calf serum and 1% Pen/Strep. The cells were passaged twice and resuspended in BD Medium #1 before seeding at 2000 cells/well on HA surfaces to which collagen VI was covalently linked, either alone or in combination with other covalently bound ECM proteins—either elastin, collagen III or vitronectin. (Cells from the two collagen VI+elastin experiments were seeded at different densities.) Time points were taken at 1 day and 6 days and were stained with calcein am dye (Live/Dead™ assay from Molecular Probes) to indicate the presence of live cells. Fluorescence microscopy images were obtained and analyzed as described in Example V. As shown in FIG. 6, covalently linked collagen VI, either alone or in combination with other covalently attached ECM proteins, was important for efficient cell adhesion and proliferation of the rat bone marrow cells.

From the foregoing description, one skilled in the art can easily ascertain the essential characteristics of this invention, and without departing from the spirit and scope thereof, can make changes and modifications of the invention to adapt it to various usage and conditions.

Without further elaboration, it is believed that one skilled in the art can, using the preceding description, utilize the present invention to its fullest extent. The preceding preferred specific embodiments are, therefore, to be construed as merely illustrative, and not limiting of the remainder of the disclosure in any way.

The entire disclosure of all applications, patents and publications cited herein are hereby incorporated by reference.

1. A surface comprising
a) a support to which is bound a cell adhesion resistant (CAR) material, and,
b) bound to the CAR material, collagen VI or a biologically active fragment or variant thereof; and, optionally,
(i) one or more different extracellular matrix (ECM) proteins, or a biologically active fragment or variant of the ECM protein, and/or
(ii) one or more polycationic polymers, or a biologically active fragment or variant of the polycationic polymer.

2. The surface of claim 1, wherein
(i) the optional ECM protein is selected from the group consisting of one or more of elastin, fibronectin, vitronectin, tenascin, laminin, entactin, aggrecan, decorin, and a collagen, and
(ii) the optional polycationic polymer is selected from the group consisting of polyethylene imine (PEI), poly-D-lysine, poly-L-lysine, poly-L-lysine, and poly-D-ornithine.

3. A surface comprising
a) a support to which is bound a CAR material, and
b) bound to the CAR material, collagen VI or a biologically active fragment or variant thereof and one or more different ECM proteins, or a biologically active fragment or variant of the ECM protein.

4. The surface of claim 3, wherein the ECM protein of is selected from the group consisting of one or more of elastin, fibronectin, vitronectin, tenascin, laminin, entactin, aggrecan, decorin, or a collagen.

5. The surface of claim 1 or 3, wherein the CAR material is selected from the group consisting of hyaluronic acid (HA) or a derivative thereof, alginic acid (AA) or a derivative thereof, poly-HEMA, polyethylene glycol (PEG), glyme or a derivative thereof, polypropylacrylamide, polyisopropylacrylamide, or a combination thereof.

6. The surface of claim 1 or 3, wherein collagen VI and/or one of more of the ECM proteins is covalently bound to the CAR material.

7. The surface of claim 6, wherein the CAR material is HA.

8. The surface of claim 1 or 3, wherein one or more of the collagen VI or the ECM proteins is non-covalently bound to the CAR material.

9. The surface of claim 8, wherein the CAR material is HA.

10. The surface of claim 1 or 3, wherein the support is a natural or synthetic organic polymer, or an inorganic composite.

11. The surface of claim 10, wherein the support is selected from the group consisting of polystyrene, polypropylene, polyethylene, polyethylene terephthalate, polytetrafluoroethylene, polylactide, cellulose, glass, or a ceramic.

12. The surface of claim 11, wherein the support is polystyrene.

13. A surface comprising
(a) a polystyrene support to which is bound hyaluronic acid, and
(b) covalently bound to the hyaluronic acid is collagen VI, or a biologically active fragment or variant thereof.

14. An article comprising a surface of claim 1.

15. The article of claim 14, which is a slide, a multi-well plate, a culture dish, a culture flask or a culture bottle.

16. The article of claim 14, which is part of a medical device, a scaffold or template for generating a 3D implant, a tissue and/or an organ, a foam, or a fiber mesh.

17. A method of making a surface of claim 1, comprising
(a) binding a CAR material to the support, and
(b) binding to the CAR material, collagen VI or a biologically active fragment or variant thereof; and optionally,
(i) one or more different ECM proteins, or a biologically active fragment or variant of the ECM protein, and/or
(ii) one or more polycationic polymers, or a biologically active fragment or variant thereof.

18. The method of claim 17, wherein the CAR material is bound to the support by
a) treating the support with an oxidizing plasma, and binding the CAR material to the treated support, or
b) treating the support with an oxidizing plasma, exposing the treated support to a polycationic polymer with amino groups to form an intermediate layer, and binding the CAR material to the intermediate layer.

19. The method of claim 18, wherein the polycationic polymer is polyethyleneimine (PEI) or poly-L-lysine (PLL).

20. A method of promoting the attachment, survival, and/or proliferation of a cell in culture, comprising contacting the cell in a culture medium with a surface comprising
(a) a support to which is bound a CAR material, and,
(b) bound to the CAR material, collagen VI, or a biologically active fragment or variant thereof; and, optionally,
(i) one or more different ECM proteins, or a biologically active fragment or variant of the ECM protein, and/or
(ii) one or more polycationic polymer,
under conditions effective for the attachment, survival, proliferation of the cell.

21. The method of claim 20, wherein
(i) the optional ECM protein is selected from the group consisting of one or more of elastin, fibronectin, vitronectin, tenascin, laminin, entactin, aggrecan, decorin, and a collagen, and
(ii) the optional polycationic polymer is selected from the group consisting of polyethylene imine (PEI), poly-D-lysine, poly-L-lysine, poly-L-lysine, and poly-D-ornithine.

22. A method of promoting the attachment, survival, and/or proliferation of a cell in culture, comprising contacting the cell in a culture medium with a surface comprising
(a) a support to which is bound a CAR material, and,
(b) bound to the CAR material, collagen VI, or a biologically active fragment or variant thereof; and one or more different ECM proteins, or a biologically active fragment or variant of the ECM protein, which ECM protein is selected from the group consisting of elastin, fibronectin, vitronectin, tenascin, laminin, entactin, aggrecan, decorin, and a collagen,
under conditions effective for the attachment, survival and/or proliferation of the cell.

23. The method of claim 20, wherein the collagen VI, one or more ECM proteins, or the polycationic polymer is covalently bound to the CAR material.

24. The method of claim 20, wherein the collagen VI, one or more ECM proteins, or the polycationic polymer is noncovalently bound to the CAR material.

25. The method of claim 23, wherein the CAR material is HA.

26. The method of claim 21, wherein the cell is a mammalian cell.

27. The method of claim 26, wherein the mammalian cell is a human cell.

28. The method of claim 26, wherein the mammalian cell is a rodent cell.

29. The method of claim 26 wherein the mammalian cell is a bone cell or a liver cell.

30. The method of claim 29 wherein the liver cell is a primary hepatocyte, a cell from an established hepatocyte cell line, a cell from a liver tumor cell line or an epithelial stem cell.

31. The method of claim 26 wherein the mammalian cell is a human liver cell of the HepG2 liver tumor cell line.

32. The method of claim 21, which is a method of promoting the survival of a primary hepatocyte or primary bone marrow cell.

33. The method of claim 21, which is a method of promoting the proliferation of a cell from an established hepatocyte or liver tumor cell line.

34. The method of claim 21, which is a method of promoting the proliferation of an epithelial stem cell.

35. The method of claim 21, wherein the culture medium is serum-free.

36. The method of claim 21, wherein the culture medium is supplemented with serum.

37. The method of claim 21, wherein the culture medium is BD Medium #1.

38. A method for identifying a test sample containing a factor that stimulates or inhibits proliferation of cells in culture, comprising
(a) adding the test sample to cultured cells incubated in serum-free medium, the cells being in contact with the surface of claim 1,
(b) measuring cell proliferation in (a), and
(c) comparing cell proliferation in (a) to proliferation of similar cells in a control culture to which the test sample has not been added,
wherein
(i) increased cell proliferation in (a) compared to the control culture indicates the presence in the test sample of a factor that stimulates cell proliferation, and
(ii) decreased proliferation in (a) compared to the control culture indicates the presence in the sample of a factor that inhibits cell proliferation.

39. A method to determine the effect of an agent on a property or behavior of the cell, comprising
(a) adding the agent to cultured cells incubated in serum-free medium, the cells being in contact with the surface of claim 1,
(b) measuring cell proliferation in (a), and
(c) comparing cell proliferation in (a) to proliferation of similar cells in a control culture to which the agent has not been added,
wherein
(i) increased cell proliferation in (a) compared to the control culture indicates that the agent stimulates cell proliferation, and
(ii) decreased proliferation in (a) compared to the control culture indicates that the agent inhibits cell proliferation.

40. The method of claim 39 wherein the agent is a drug.

41. The method of claim 39 wherein the agent is a small molecule.

42. A kit useful for promoting the attachment, survival, and/or proliferation of cells, comprising a surface according to claim 1 and one or more components or reagents suitable for culturing the cells and enabling cell attachment, survival, and/or proliferation.

43. A kit for identifying a factor that modulates positively or negatively proliferation of cells in culture, comprising the surface of claim 1 and one or more components or reagents suitable for (a) growing the cells and (b) measuring cell proliferation.
